(19) United States
(12) Patent Application Publication
(10) Pub. No.: US 2002/0123785 A1
(43) Pub. Date: Sep. 5, 2002

(54) Cardiac lead permitting easy extraction

(75) Inventors: Yongxing Zhang, Little Canada, MN (US); Michael P. Campbell, New Brighton, MN (US); J. Michael Hoch, Plymouth, MN (US); John S. Greenland, San Diego, CA (US)

Correspondence Address: NIKOLAI MERSEREAU & DIETZ, P.A., 820 INTERNATIONAL CENTRE, 900 SECOND AVENUE SOUTH, MINNEAPOLIS, MN, 55402

(73) Assignee: Cardiac Pacemakers, Inc., Sanit Paul, MN (US)

(21) Appl. No.: 09/797,926

(22) Filed: Mar. 2, 2001

(51) Int. Cl. A61N 1/05

(52) U.S. Cl. 607/126

(57) ABSTRACT

An extractable lead and method for lead explantation is provided. The lead includes a distal element having a cavity. The cavity shaped to receive the distal end of a removal stylet. The cavity and the distal end configured to cooperate such that the distal end is secured within the cavity by rotation of the stylet. The method provides for removal of an implanted lead by securing a distal end of a removal stylet with a cavity by rotation and applying an extracting force to the lead through the removal stylet.

### BACKGROUND OF THE INVENTION

1. Field of the Invention

The present invention relates generally to lead extraction and, more particularly, to a method and apparatus for lead extraction using a removal stylet.

2. Background of the Related Art

Cardiac leads are implanted in the heart of patients with arrhythmias, patients with CHF, and patients subject to cardiac fibrillations. The leads are typically permanent being placed and secured at a particular location within the heart or an adjacent vein. Over time, the lead can become encapsulated in a scar tissue, particularly the tip electrode, further securing the lead. In certain circumstances, the lead may need to be removed such as, when a lead becomes inoperative, when there is an infection in or around the lead or a portion of a lead, or when the lead is no longer needed. If left in the patient's heart, the lead may reduce the efficiency of the heart valves through which it passes. The efficiency is further reduced when an additional lead is required to replace the function of the inoperable lead. In patients with poor valve function or with hearts that otherwise operate inefficiently, further impairing the valve function is at best a poor option.

Extraction of chronically implanted leads is a difficult procedure. The lead is fragile and is typically firmly embedded in scar tissue. Removal can be accomplished by open-heart surgery, but open-heart surgery is complicated, risky and costly. Intravascular countertraction techniques may also be used. Intravascular countertraction techniques typically use locking stylets, sheaths, snares and retrieval baskets depending upon the type and location of the implanted lead.

The leads typically include a coiled electrode, an insulating cover, and one or more electrodes at the lead's distal end. Because the electrode is embedded in scar tissue, the force required to remove the electrode by countertraction can be relatively large. In some situations, the countertraction apparatus cannot confer adequate force to displace the tip electrode from the scar tissue leaving the lead embedded in the patient's heart. In other situations, the intravascular countertraction technique can actually pull apart the lead body. The body is pulled apart because most intravascular countertraction techniques apply the removal force to the lead body proximal the embedded distal end. Typically the lead breaks distal to the point where the countertraction apparatus applies force. Thus, a portion of the broken lead is left embedded in the patient's heart. The breaking of the lead is particularly troublesome when only the insulator is broken and the conductor is uncoiled within the patient. The exposed end of a coiled wire conductor, once extended and stretched or during the process of extending and stretching, presents a risk of cutting into the adjacent tissues.

Some prior methods for extraction involved inserting a hollow tube with a beveled tip that engaged the coil of the implanted electrode. However, if the lead could not be removed because of some complication, the tip of the tool is nevertheless locked in place and cannot be removed from the lead. Consequently, both the removal tool and the lead will have to be surgically removed. Other methods, employ a stylet that is inserted internally through a lumen within the lead having a tip configured to engage the inner surface of the lumen. In addition to the removal problems of the tubular removal tubes, the intralumenal removal tubes

Another method for extracting leads involves the manual manipulation without the use of a tube, stylet, or other tool. Such methods are typically inapplicable when the lead has become encapsulated by scar tissue. Moreover, the method puts excessive strain and tension on the insulation material of the lead. Should the insulation break the conductor could become uncoiled leading to possible damage of the heart and blood vessels by the thin sharp wires. Should the lead break, the broken inner coil and insulation could damage the heart or surrounding blood vessels and surgical removal of the broken lead would be required.

The present invention meets the needs above need and provides additional improvements and advantages that will be recognized by those skilled in the art upon review of the following drawings and description.

### SUMMARY OF THE INVENTION

The present invention provides an apparatus and method permitting extraction of an implanted lead. The invention provides a distal element that allows a removal stylet to be removably secured within the distal element. A extractable lead in accordance with the present invention includes a lead body and a distal element. The lead body may have a lumen. The distal element defines a cavity at the proximal end of the distal element. The cavity is configured to receive a distal end of a removal stylet. The distal end of the stylet is secured within the cavity by rotating the stylet relative to the distal element. The distal end of the stylet may be in the form of a screw helix to be received in a threaded cavity. Alternatively, the distal end of the removal stylet may include one or more grooves and the cavity may include one or more protuberances shaped to cooperate with the grooves to rotatably secure the removal stylet to the distal element. In another alternative embodiment, the distal end of the removal stylet may include one or more arms and the cavity may be configured to lock the one or more arms within cavity upon rotation of the stylet. To lock the arms, the cavity may be configured with a pin bisecting the cavity to rotatably receive the at least one arm of the stylet.

A lead in accordance with the present invention may be explanted by securing the distal end of the removal stylet within the cavity. The stylet is inserted through a lumen within the lead or if the lead does not have a lumen through an insertion catheter place over the lead body. Once the distal end of the stylet is positioned adjacent the proximal end of the distal element, the distal end of the stylet is rotatably secured within the cavity. A force is then applied to remove the implanted lead.

### BRIEF DESCRIPTION OF THE DRAWINGS

FIG. 1 illustrates the cooperation of an embodiment of the distal portion of the lead removal tool with the distal element of the lead body;

FIG. 2 illustrates a greatly enlarged partial side view of a lead removal tool in accordance with the present invention;

FIG. 3A illustrates a partial side view of an embodiment of the mating structure in accordance with the present invention;

FIG. 3B illustrates an end view of the embodiment of the mating structure in accordance as shown in FIG. 3A;

FIG. 3C illustrates a partial side view of another embodiment of the mating structure in accordance with the present invention;

FIG. 3D illustrates an end view of the embodiment of the mating structure in accordance as shown in FIG. 3C; and

FIGS. 4A, 4B and 4C illustrate partial side view of embodiments of leads in accordance with the present invention.

### DETAILED DESCRIPTION OF THE INVENTION

An extractable lead in accordance with the present invention is applicable to removal of a variety of implanted leads. The invention is described in the context a cardiac lead as a specific example for illustrative purposes only. The appended claims are not intended to be limited to any specific example or embodiment described in this patent. It will be understood by those skilled in the art that the present invention may be used to explant a wide variety of leads including, but not limited to, sensing leads, unipolar leads, multipolar leads, and leads with extendable screw helix positive fixation electrodes. Further, in the drawings described below, the reference numerals are generally repeated where identical elements appear in more than one figure.

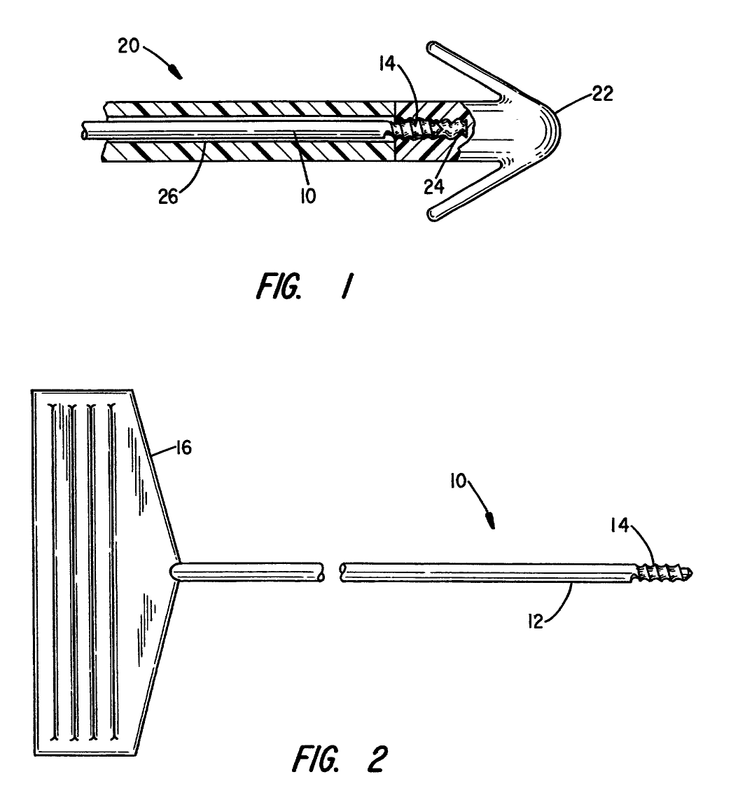

FIG. 1 illustrates a lead removal stylet 10 in accordance with the present invention. Removal stylet 10 includes an elongated body 12 having a mating structure 14 at a distal end of the removal stylet. Removal stylet 10 may also include a gripping portion 16 at the proximal end of the removal stylet for gripping with fingers, forceps or other tools. Gripping structure 16 is typically configured to allow the user to apply a rotational and a pulling force to removal stylet 10 while the removal stylet is residing in the lumen of a lead. When a gripping structure is not provided, forces are conferred upon the removal stylet by manipulation of a proximal region of the stylet with fingers or a gripping device, such as forceps or pliers. Removal stylet 10 is typically composed of a flexible metal or polymeric material having sufficient tensile strength to permit dislodgment of an electrode. Further, body 12 and mating structure 14 are diametrically sized to slidably fit through the lumen of a lead. The precise choice of diametric size will vary with the internal diameter of the lumen and structure of the lead being removed. Mating structure 14 is further configured to cooperate with a cavity on the proximal end of a distal electrode to allow dislodgement and/or removal of a lead from a patient.

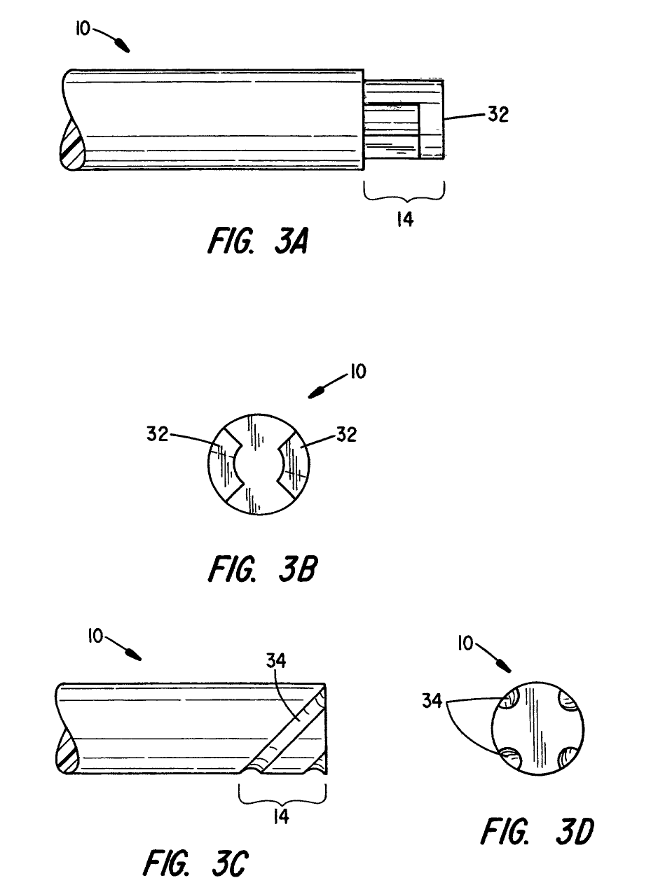

FIG. 2 illustrates the cooperation of removal stylet 10 with a lead 20. In the embodiment shown, removal stylet 10 is inserted into lead 20 through an internal lumen 26 which guides stylet 10 to a distal element 22. Distal element 22 may be an electrode, a sensor, a drug eluting element, or any structure defining a cavity 24 shaped to receive mating structure 14. Mating structure 14 is securedly received within cavity 24 by rotating mating structure 14 about the longitudinal axis of the removal stylet. Typically, mating structure 14 is further configured to allow removal by rotating mating structure 14 in the opposite direction. Cavity 24 is generally configured to cooperate with mating structure 14 to secure the proximal end of a distal electrode. In the embodiment shown in FIG. 2, the mating structure is in the form of a screw helix and cavity 22 is threaded to securedly receive the screw helix. FIGS. 3A, 3B, 3C and 3D illustrate alternative configurations for mating structure 14 in accordance with the present invention. FIGS. 3A and 3B illustrate side and end views of an embodiment of the present invention. Mating structure 14 of the embodiment in FIGS. 3A and 3B includes at least one arm 32 configured to lock within cavity 24 upon rotation. The distal portions of arms 32 are angled to facilitate the locking within the cavity. To receive arms 32, cavity 24 may be provided with a pin bisecting the cavity that is received by the arms to lock the mating structure within the cavity. Alternatively, cavity 24 may be shaped to receive arms 32 to rotatably lock mating structure 14 within the cavity, as will be recognized by those skilled in the art. FIGS. 3C and 3D illustrate side and end views of another embodiment of the present invention. Mating structure 14 of the embodiment in FIGS. 3C and 3D includes at least one channel 36 configured to lock within cavity 24 upon rotation. Channels 36 are typically angled to facilitate the locking within the cavity. In the particular embodiment of the channels shown in FIGS. 3C and 3D, channels 36 are spirally wound in parallel along the longitudinal axis of removal stylet 10. To receive channels 36, cavity 24 may be provided with one or more protuberances configured to be received by the one or more channels 34 to rotatably lock the mating structure within cavity 24. The protuberances may be elongate conforming to the channels along there entire length or they may extend over only a portion of the channel. Each of the alternative configurations for mating structures 14 will have a variety of respective cavities 24 shaped to rotatably receive the particular mating structure as will be recognized by those skilled in the art.

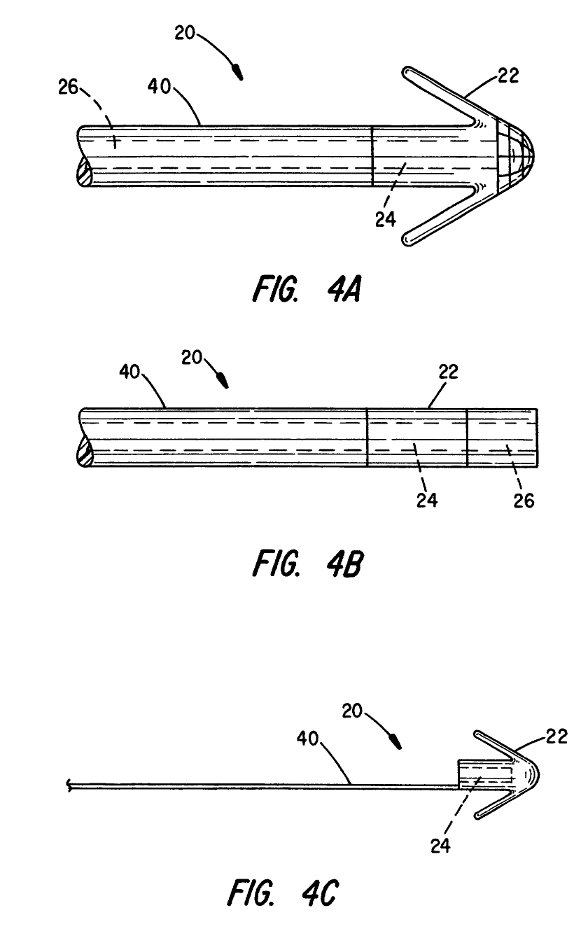

FIGS. 4A, 4B and 4C illustrate various lead configurations in accordance with the present invention. The specific leads shown are for exemplary purposes only and are in no way intended to limit the scope of the claims. FIG. 4A illustrates a terminal electrode having passive fixation tines and defining a cavity 24. A lead body 40 includes a lumen 26 that communicates with cavity 24. When a removal stylet is inserted into lumen 26 at the lumen's proximal end, the lumen functions to guide mating structure 14 to cavity 24 to be secured. FIG. 4B illustrates a ring electrode 22 located proximal to the distal end of the lead body. Ring electrode 22 defines a cavity 24 that extends through ring electrode 22. Lumen 26 may also extend beyond ring electrode 22 allowing the delivery of fluids through the lumen and into the patient and may also allow for over-the-wire implantation. FIG. 4C illustrates a lead having a reduced diameter lead body 40 without a lumen. In the configuration of FIG. 4C, a guide catheter is also provided to guide the removal stylet to cavity 24 on electrode 22. The guide catheter includes a lumen that receives connector pin 48 and is then guided to electrode 22 along lead body 40. Once positioned adjacent to or having received electrode 22, removal stylet 14 is also passed through the lumen until it is adjacent to and then received by cavity 24.

In use, the lead and removal stylet of the present invention work in concert to facilitate removal of the implanted lead. The lead typically enters the circulatory system through the subclavian vein. For leads having a configuration similar to those shown in FIGS. 2, 4A and 4B, the distal end of removal stylet is inserted through the leads lumen and advanced until the mating structure reaches the cavity of the distal element. The stylets with mating structures as shown in FIGS. 1, 2, 3C and 3D are then rotated such that the screw helix or channel draws the mating structure into the particular distal element. For mating structures more in accordance with that of FIGS. 3A and 3B, the mating structure is advanced into the cavity and then rotated to secure the removal stylet to the distal element.

What is claimed is:

1. An extractable lead, comprising:
a lead body; and
a distal element defining a cavity wherein the cavity is configured to receive a distal end of a removal stylet so as to rotatably secure the distal end within the cavity.

2. An extractable lead, as in claim 1, wherein the lead body further comprises a lumen.

3. An extractable lead, as in claim 1, wherein the cavity is threaded and the distal end of the removal stylet is a screw helix.

4. An extractable lead, as in claim 1, wherein the cavity includes at least one protuberance and the distal end of the removal stylet includes at least one groove shaped to receive the at least one protuberance.

5. An extractable lead, as in claim 1, wherein the distal end of the removal stylet include at least one arm and the cavity is configured to lock the at least one arm within cavity upon rotation of the stylet.

6. An extractable lead, as in claim 5, wherein the cavity is configured with a pin bisecting the cavity to rotatably receive the at least one arm of the stylet.

7. An extractable lead, comprising a means for securably receiving a distal end of a removal stylet.

8. A method for removing an implanted lead, comprising:
guiding a removal stylet to a proximal end of a distal element of the implanted lead, the stylet comprising a distal end configured to be removably secured by rotatation within a cavity at the proximal end of the distal element;
securing the distal end of the removal stylet within the cavity
removing the implanted lead.

9. A method, as in claim 8, wherein the removal stylet is guided to the distal element by passing the removal stylet through a lumen.

10. A method, as in claim 9, wherein the lumen is defined by the lead body.

11. A method, as in claim 9, wherein the lumen is defined by a guide catheter.

12. A method for removing an implanted lead, comprising:
a step for guiding a removal stylet to a cavity defined by a proximal end of a distal element;
a step for securing the distal end of the removal stylet within the cavity; and
a step for removing the implanted lead.
